(19) United States
(12) Patent Application Publication
(10) Pub. No.: US 2020/0268299 A1
(43) Pub. Date: Aug. 27, 2020

(54) STATE DISPLAY APPARATUS, STATE DISPLAY SYSTEM, AND NON-TRANSITORY COMPUTER READABLE MEDIUM STORING PROGRAM

(71) Applicant: FUJI XEROX CO., LTD., Tokyo (JP)

(72) Inventor: Kazuhide KOBAYASHI, Kanagawa (JP)

(73) Assignee: FUJI XEROX CO., LTD., Tokyo (JP)

(21) Appl. No.: 16/517,671

(22) Filed: Jul. 22, 2019

(30) Foreign Application Priority Data
Feb. 22, 2019 (JP) 2019-030486

(51) Int. Cl. A61B 5/16; A61B 5/00; A61B 5/024

(52) U.S. Cl. CPC A61B 5/165; A61B 5/486; A61B 5/02405; A61B 5/681

(57) ABSTRACT

A state display apparatus includes a display section that displays a state of a measurement target subject based on biometric data of the measurement target subject; and a control section that performs control such that a display mode at the time of displaying a state of the measurement target subject next time onwards is changed so as to be different from a display mode at the time of displaying a first state based on first biometric data of the measurement target subject, in a case where second biometric data acquired at the time of displaying the first state on the display section satisfies a predetermined condition, at the time of performing control such that the display section displays the first state.

### CROSS-REFERENCE TO RELATED APPLICATIONS

This application is based on and claims priority under 35 USC 119 from Japanese Patent Application No. 2019-030486 filed Feb. 22, 2019.

### BACKGROUND

### (i) Technical Field

The present invention relates to a state display apparatus, a state display system, and a non-transitory computer readable medium storing a program.

### (ii) Related Art

JP5492247B discloses an indicator generating device. In the indicator generating device, frequency analysis is performed on a heart rate measurement value and a heart rate interval measurement value of a subject so as to obtain a high frequency index, a low frequency index, and a ratio thereof. A sympathetic nervous indicator, a parasympathetic nervous indicator and a heart rate are calculated from the result of the frequency analysis in three states: a resting state, a state for executing the random number generation task, and the resting state after task execution. The sympathetic nervous indicator, the parasympathetic nervous indicator, and the heart rate calculated for each state are estimated in a multiple comparison with a threshold value which is set based on the sympathetic nervous indicators and the parasympathetic nervous indicators of normal persons calculated in advance and stored.

JP5958825B discloses an affectivity estimation system for accurately estimating the affectivity of a subject from biological information. The affectivity estimation system includes: an acquisition unit, a biometric data analysis unit, a positive/negative analysis unit, and a comprehensive estimation unit. The acquisition unit acquires responses of a sympathetic nervous system and a parasympathetic nervous system as biometric data. The biometric data analysis unit determines a candidate group of affectivity factors of the subject based on the acquired biometric data. The positive/negative analysis unit determines whether the internal state of the subject is comfort or discomfort based on information obtained from the subject. The comprehensive estimation unit comprehensively estimates affectivity by integrating the analysis results of the biometric data analysis unit and the positive/negative analysis unit.

### SUMMARY

Aspects of non-limiting embodiments of the present disclosure relate to a state display apparatus, a state display system and a non-transitory computer readable medium storing a program capable of preventing the state of the measurement target subject from being deteriorated by displaying the state of the measurement target subject in a case of displaying the state of the measurement target subject based on the biometric data of the measurement target subject.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present invention, there is provided a state display apparatus including: a display section that displays a state of a measurement target subject based on biometric data of the measurement target subject; and a control section that performs control such that a display mode at the time of displaying a state of the measurement target subject next time onwards is changed so as to be different from a display mode at the time of displaying a first state based on first biometric data of the measurement target subject, in a case where second biometric data acquired at the time of displaying the first state on the display section satisfies a predetermined condition, at the time of performing control such that the display section displays the first state.

### BRIEF DESCRIPTION OF THE DRAWINGS

Exemplary embodiment(s) of the present invention will be described in detail based on the following figures, wherein:

FIG. 1 is a diagram illustrating a configuration of an indicator display system according to an embodiment of the present invention;

FIG. 2 is a block diagram illustrating a hardware configuration of a management server 14 according to an embodiment of the present invention;

FIG. 3 is a diagram illustrating a functional configuration of the management server 14 according to an embodiment of the present invention;

FIG. 4 is a diagram illustrating an example of user information stored in a data storage unit 33 of the management server 14;

FIG. 5 is a flowchart for explaining an operation of the management server 14;

FIG. 6 is a diagram illustrating an example of a display screen showing a level of stress of the measurement target subject in the portable terminal device 12;

FIGS. 7A and 7B are diagrams illustrating an example of variation in level of stress of the measurement target subject;

FIG. 8 is a diagram illustrating a modification example of the display screen showing the level of stress of the measurement target subject in the portable terminal device 12 illustrated in FIG. 6;

FIGS. 9A, 9B, and 9C are diagrams illustrating modification examples of the display screen in the portable terminal device 12 illustrated in FIG. 6;

FIG. 10 is a diagram illustrating a modification example of the display screen showing the level of stress of the measurement target subject in the portable terminal device 12; and

FIGS. 11A and 11B are diagrams illustrating modification examples of the display screen in the portable terminal device 12 illustrated in FIG. 10.

### DETAILED DESCRIPTION

Hereinafter, exemplary embodiments of the present invention will be described in detail with reference to the accompanying drawings.

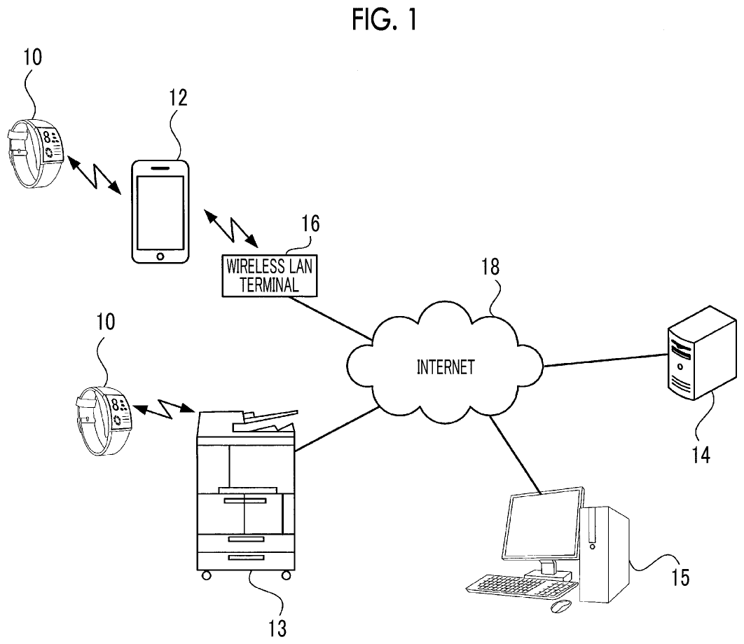

FIG. 1 is a diagram illustrating a configuration of an indicator display system which is an example of a state display system according to an embodiment of the present invention.

As illustrated in FIG. 1, the indicator display system according to the embodiment of the present invention is composed of: a wearable terminal device 10 as a detection device; a management server 14 that is connected to the wearable terminal device 10 through the Internet 18 by using a portable terminal device 12 such as a smartphone or a tablet terminal or the image forming apparatus 13 such as a multifunction peripheral; and a terminal device 15 such as a personal computer connected to the management server 14 through the Internet 18. The wearable terminal device 10, the portable terminal device 12, the image forming apparatus 13, and the terminal device 15 each also have a function as a display apparatus for displaying contents and the like of notification from the management server 14.

The wearable terminal device 10 is a device worn by a user to detect biometric data such as fluctuation in heart rate, and has, for example, a heart rate meter (which may be a pulse meter) for detecting fluctuation in heart rate of a user and an accelerometer for detecting acceleration. The wearable terminal device 10 is, for example, a wrist type, and is worn on an arm of a measurement target subject to detect biometric data of the measurement target subject. The wearable terminal device 10 is mutually connected to the portable terminal device 12 or the image forming apparatus 13 by near field wireless communication such as Bluetooth (registered trademark) to transmit and receive information.

The portable terminal device 12 is connected to a wireless LAN terminal 16 through a wireless network, and thereby connected to the management server 14 through the Internet 18.

The image forming apparatus 13 is connected to the management server 14 through the Internet 18. Further, the image forming apparatus 13 may be connected to the management server 14 through a wireless or wired network.

That is, the wearable terminal device 10 transmits the detected biometric data to the portable terminal device 12 or the image forming apparatus 13 by near field wireless communication, and the portable terminal device 12 and the image forming apparatus 13 transmit the detected biometric data to the management server 14 through the Internet 18.

The management server 14 acquires biometric data of the measurement target subject from the wearable terminal device 10, and estimates the stress state of the measurement target subject from the biometric data. Then, for example, control for notifying and displaying the estimation result to the portable terminal device 12 of the measurement target subject, the wearable terminal device 10 of the measurement target subject, the image forming apparatus 13, and the terminal device 15 of an administrator or the measurement target subject is performed.

Here, it is known that the sympathetic nerve is dominant in a case where the subject of measurement is in a high stress state, and the parasympathetic nerve is dominant in a case where the measurement target subject is in a low stress state. Therefore, in the indicator display system of the present embodiment, the stress state of the measurement target subject is estimated using the level of stress calculated based on the value of LF/HF, which is a sympathetic nerve activity indicator, from the biometric data detected from the measurement target subject.

In the present embodiment, for example, the predetermined value of LF/HF is set to 100% stress, and the value of LF/HF of the measurement target subject is converted as a percentage so as to estimate the value as the level of stress of the measurement target subject. In addition, the predetermined value of LF/HF, which is set to 100% stress, is converted based on a plurality of divided levels and estimated as a level of stress. For example, the predetermined value of LF/HF, which is set to 100% stress, is converted based on three levels, and a first threshold value and a second threshold value are set. Then, a state where the level of stress is low in a case where the value converted as the level of stress is equal to or less than the first threshold value is estimated as a low stress state, a state where the value is greater than the first threshold value and not greater than the second threshold value is estimated as a medium stress state, and a state where the level of stress is greater than the second threshold value is estimated as a high stress state. Then, in a high stress state where the level of stress is high, the portable terminal device 12 of the measurement target subject is notified. The level of stress of the measurement target subject may be notified to the portable terminal device 12 of the measurement target subject at a preset time.

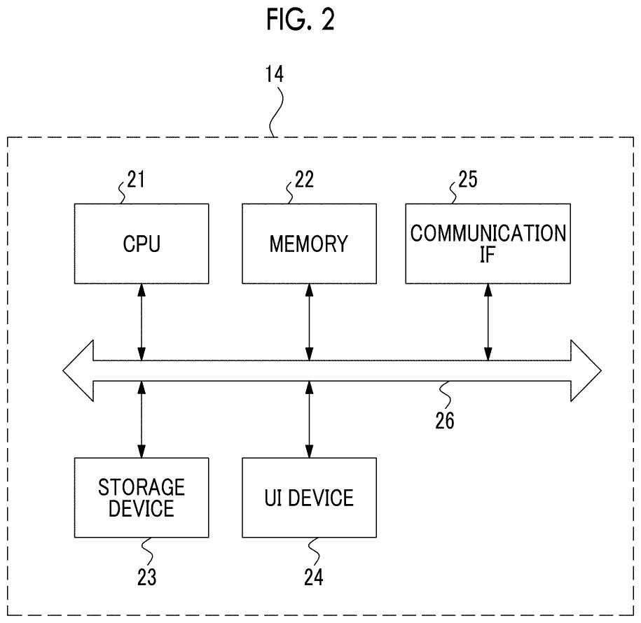

Next, a hardware configuration of the management server 14 in the indicator display system of the present embodiment is illustrated in FIG. 2.

As illustrated in FIG. 2, the management server 14 is provided with a CPU 21, a memory 22 capable of temporarily storing data, a storage device 23 such as a hard disk drive (HDD), a user interface (UI) device 24 including a touch panel or a liquid crystal display and a keyboard, and a communication interface (IF) 25 that transmits and receives data between an external device and the like. These components are connected to one another through a control bus 26.

The CPU 21 executes predetermined processing based on a control program stored in the memory 22 or the storage device 23, thereby controlling an operation of the management server 14. In the present embodiment, the above description is given of the CPU 21 reading and executing the control program stored in the memory 22 or the storage device 23. However, it is possible to provide the program in the CPU 21 in state where the program is stored in a storage medium such as a CD-ROM.

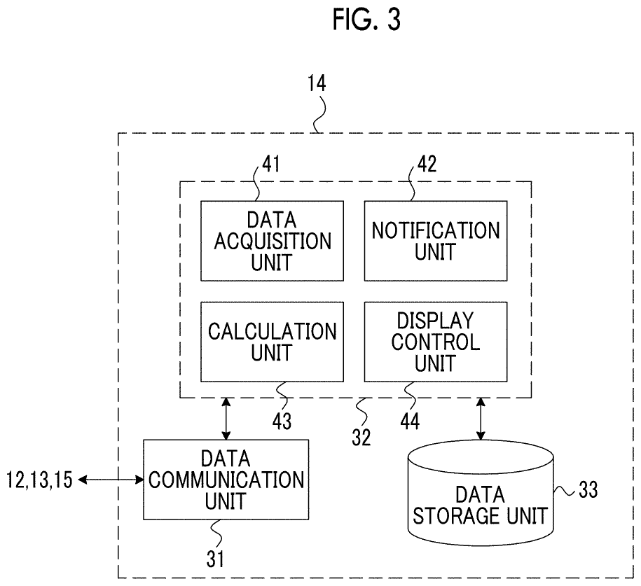

FIG. 3 is a block diagram illustrating a functional configuration of the management server 14 realized by executing the control program described above.

As illustrated in FIG. 3, the management server 14 of the present embodiment includes a data communication unit 31, a control unit 32, and a data storage unit 33.

The data communication unit 31 performs data communication with the portable terminal device 12, the image forming apparatus 13, and the terminal device 15 through the Internet 18.

The control unit 32 controls an operation of the management server 14 and includes a data acquisition unit 41, a notification unit 42, a calculation unit 43, and a display control unit 44.

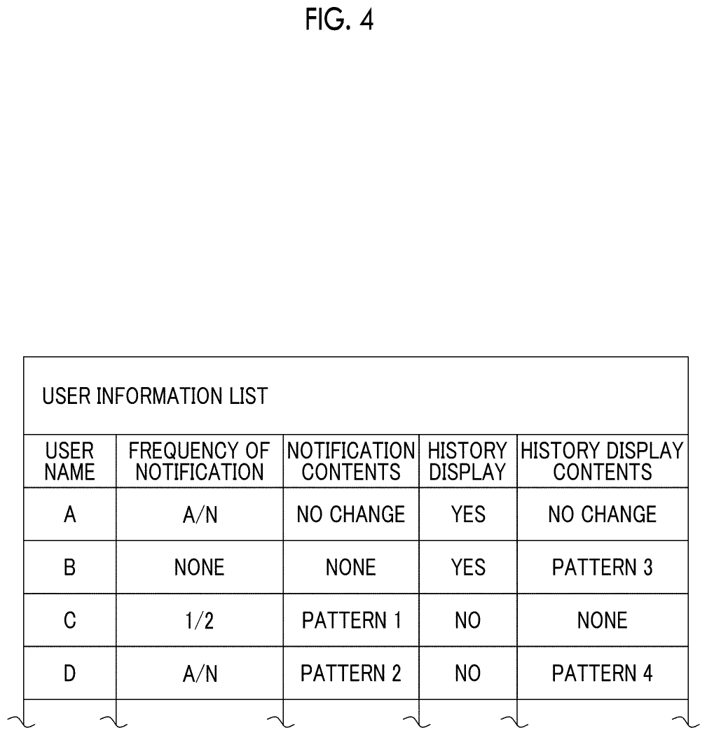

The data storage unit 33 stores various kinds of information such as user information. For example, as illustrated in FIG. 4, the data storage unit 33 stores, in association with one another, user information such as a frequency of notification for notifying the stress state of the measurement target subject, contents of notification, presence or absence of history display, history display contents, and the like, for each user as the measurement target subject. These pieces of user information are changed in accordance with the level of stress of the user who is the measurement target subject.

The data acquisition unit 41 acquires biometric data of the measurement target subject.

The calculation unit 43 calculates the value of LF/HF of the measurement target subject from the biometric data acquired by the data acquisition unit 41.

Specifically, the calculation unit 43 performs power spectrum analysis of frequency components of the RR intervals in chronological order from the biometric data acquired by the data acquisition unit 41. Then, the calculation unit 43 obtains an LF affected by the sympathetic nerve and an HF affected by the parasympathetic nerve, among the frequency components of the RR intervals in chronological order, and further calculates the value of LF/HF which is a sympathetic nerve activity indicator.

In a case where the LF/HF value calculated by the calculation unit 43 is greater than the second threshold value, the notification unit 42 determines that the measurement target subject is in a high stress state, and notifies the level of stress of the measurement target subject to the portable terminal device 12. The notification unit 42 may be set to notify the current level of stress of the measurement target subject at a preset time.

The display control unit 44 controls a screen displayed on each of the portable terminal device 12, the image forming apparatus 13, and the terminal device 15. Further, the display control unit 44 controls the display screen of the portable terminal device 12 such that the level of stress which is the state of the measurement target subject is displayed based on the value of LF/HF calculated by the calculation unit 43.

The data acquisition unit 41 controls the portable terminal device 12 such that the level of stress of the measurement target subject is displayed on the portable terminal device 12 of the measurement target subject based on the value of LF/HF calculated by the calculation unit 43. In this case, the data acquisition unit 41 acquires biometric data of the measurement target subject detected using the wearable terminal device 10.

Here, “the biometric data of the measurement target subject detected using the wearable terminal device 10 in a case where the portable terminal device 12 is controlled to display the level of stress of the measurement target subject on the portable terminal device 12 of the measurement target subject” is defined as follows. The biometric data is detected using the wearable terminal device 10 immediately after the portable terminal device 12 is controlled to display the level of stress of the measurement target subject on the portable terminal device 12 of the measurement target subject. For example, the biometric data is detected by the wearable terminal device 10 until the display disappears after the level of stress of the measurement target subject is displayed on the portable terminal device 12 of the measurement target subject. Examples of the condition that the display disappears include a case where a preset time has elapsed, a case where the accelerometer detects that the measurement target subject moves an arm, a case where the line of sight of the measurement target subject is not detected, and the like. Alternatively, the biometric data may be detected by the wearable terminal device 10 after a preset time has elapsed since the level of stress of the measurement target subject is displayed on the portable terminal device 12 of the measurement target subject. For example, the biometric data is detected by the portable terminal device 12 after three seconds since the level of stress of the measurement target subject is displayed on the portable terminal device 12 of the measurement target subject. That is, “the biometric data of the measurement target subject detected using the wearable terminal device 10 in a case where the portable terminal device is controlled to display the level of stress of the measurement target subject on the portable terminal device 12 of the measurement target subject” is biometric data detected using the wearable terminal device 10 after the measurement target subject confirms the display of the level of stress or after an estimated result is confirmed.

Here, the stress state of the measurement target subject may be deteriorated by acquiring the biometric data and causing the measurement target subject to correctly recognize the stress state.

In a case where the biometric data acquired at the time of displaying the stress state of the measurement target subject on the portable terminal device 12 of the measurement target subject satisfies a predetermined condition, the display control unit 44 performs control such that the display mode at the time of displaying the stress state of the measurement target subject on the portable terminal device 12 next time onwards is changed.

Specifically, in a case where the value of LF/HF calculated based on the biometric data acquired at the time of displaying the level of stress on the portable terminal device 12 of the measurement target subject changes by a predetermined value or more from the value of LF/HF calculated before the level of stress is displayed, the display control unit 44 performs control for changing the display mode at the time of displaying the level of stress based on the values of LF/HF calculated next time onwards on the portable terminal device 12.

Here, the phrase “control for changing the display mode” is defined to include control for not displaying the level of stress of the measurement target subject on the portable terminal device 12 of the measurement target subject.

Further, the phrase “control for changing the display mode at the time of displaying the level of stress based on the values of LF/HF calculated next time onwards on the portable terminal device 12” is defined as follows. The value of LF/HF calculated based on the biometric data acquired at the time of displaying the level of stress may change by the predetermined value or more from the LF/HF calculated before the level of stress is displayed. In this case, the display control unit 44 performs control for changing the display mode at the time of displaying the level of stress based on the value of LF/HF calculated next time by the calculation unit 43 and further changing the display mode at the time of displaying the level of stress on the portable terminal device 12 until the value of LF/HF calculated based on the biometric data acquired at the time of displaying the level of stress after changing the display mode does not change by the predetermined value or more from the value of LF/HF calculated before the display. It should be noted that the display control unit 44 may perform control for changing the display mode until the measurement target subject makes any intention for display.

In addition, for example, the “high stress state” may be displayed on the portable terminal device 12. At this time, the value of LF/HF calculated based on the biometric data detected using the wearable terminal device 10 may change by the predetermined value or more from the value of LF/HF calculated before the display. In this case, the display control unit 44 performs control for changing the contents of notification at the time at which the notification unit 42 notifies the current level of stress of the measurement target subject based on the values of LF/HF calculated next time onwards.

Here, the phrase “changing the contents of notification” is defined to include, for example: not causing the notification unit 42 to notify the portable terminal device 12 of the measurement target subject of the level of stress of the measurement target subject; changing the frequency of notification; changing display of an image of an animal, an image of a landscape, an image registered by a user in advance, and the like in order to make the sympathetic nervous system of the measurement target subject dominant; changing display for prompting the measurement target subject to make the sympathetic nervous system of the measurement target subject dominant; and the like. In addition, the phrase “prompting the measurement target subject to make the sympathetic nervous system of the measurement target subject dominant” is defined to include, for example, recommendation of taking a break, recommendation of listening to music, recommendation of going home, and the like.

The display control unit 44 performs control for displaying the current level of stress of the measurement target subject in the portable terminal device 12 together with the level of stress based on the previously calculated value of LF/HF as an indicator calculated based on the biometric data acquired before the current biometric data is acquired. Further, the display control unit 44 performs control for consecutively displaying the level of stress based on a plurality of previously calculated values of LF/HF as an indicator calculated based on the biometric data acquired before the current biometric data is acquired and the current level of stress of the measurement target subject in the portable terminal device 12.

In addition, the value of LF/HF is calculated based on the biometric data acquired at the time of consecutively displaying the level of stress based on the plurality of previously calculated values of LF/HF and the current level of stress of the measurement target subject in the portable terminal device 12. The value of LF/HF may change by the predetermined value or more from the value of LF/HF calculated before display. In this case, the display control unit 44 performs control for changing the display, which is performed at the time of displaying the level of stress based on the values of LF/HF calculated next time onwards on the portable terminal device 12, to a display which makes deterioration in the stress state of the measurement target subject less noticeable.

Here, “the display which makes deterioration in the stress state of the measurement target subject less noticeable” is defined to include, for example: a display in which the current level of stress of the measurement target subject is displayed in the same color arrangement as the level of stress based on the previously calculated value of LF/HF; and a display in which the current level of stress of the measurement target subject is displayed at a scale at which the difference with between the current level of stress and the level of stress based on the previously calculated value of LF/HF is less noticeable. The display in the same color arrangement is defined to include, for example, a display in which the previous level of stress is displayed in light blue and a display in which the current level of stress is displayed in blue.

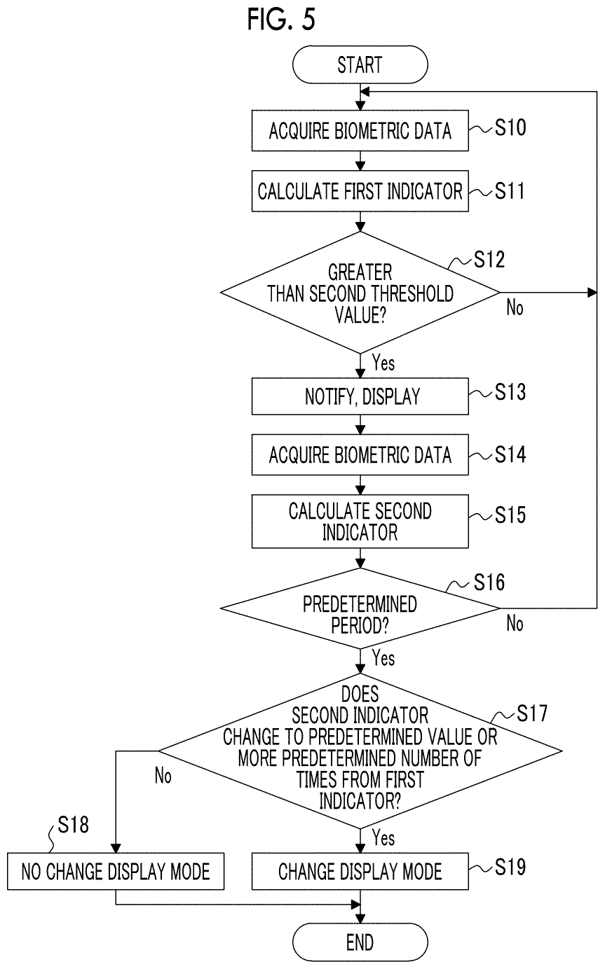

Next, the operation of the management server 14 in the indicator display system of the present embodiment will be described with reference to FIGS. 5 to 8.

First, in step S10, the management server 14 causes the data acquisition unit 41 to acquire the biometric data of the measurement target subject.

Then, in step S11, the management server 14 causes the calculation unit 43 to calculate the value of LF/HF as a first indicator, which is an activity ratio of the sympathetic nerve and the parasympathetic nerve, based on the acquired biometric data.

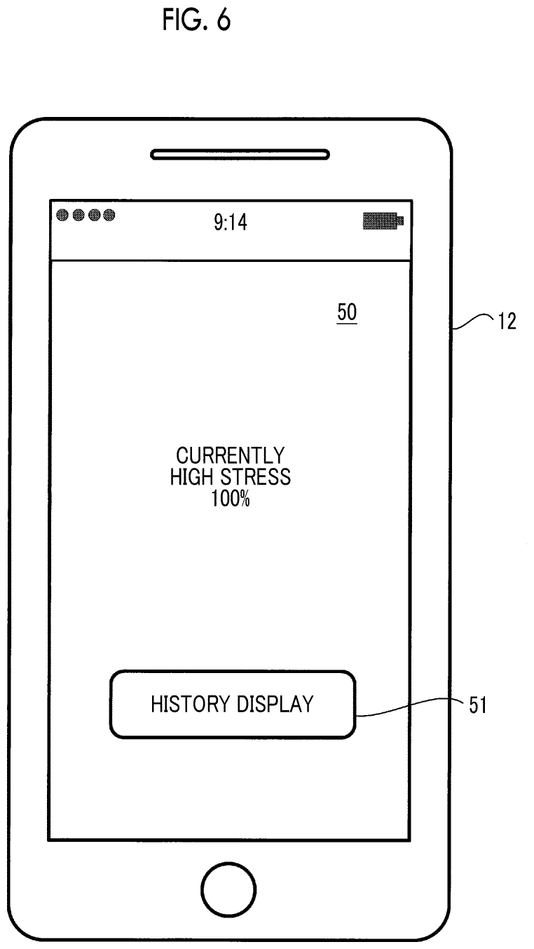

Then, in step S12, it is determined whether or not the value of LF/HF calculated in step S11 is greater than the second threshold value. Then, in a case where the value of LF/HF is equal to or less than the second threshold value, the process returns to step S10. In a case where the value of LF/HF is greater than the second threshold value, the notification unit 42 notifies the level of stress based on the value of LF/HF of the measurement target subject to the portable terminal device 12 of the measurement target subject in step S13. Then, for example, as illustrated in FIG. 6, the display control unit 44 displays the current level of stress which is in a high stress state on the display screen of the portable terminal device 12 of the measurement target subject.

Then, in step S14, the data acquisition unit 41 acquires the biometric data detected using the wearable terminal device immediately after the portable terminal device 12 is controlled such that the display is performed on the portable terminal device 12 of the measurement target subject in step S13.

Then, in step S15, the value of LF/HF as a second indicator is calculated from the biometric data acquired by the data acquisition unit 41 in step S14.

Then, in step S16, steps S10 to S15 are repeated for a predetermined period.

Then, in step S17, the biometric data of the measurement target subject and the data of the value of LF/HF calculated based on the biometric data are accumulated. In this case, it is determined whether or not the value of LF/HF as the second indicator, which is calculated after the display screen displays the level of stress based on the first indicator, changes by the predetermined value or more a predetermined number of times from the value of LF/HF calculated before the level of stress based on the first indicator is displayed. For example, it is determined whether or not the value of LF/HF calculated after the display of the level of stress on the display screen changes by a predetermined value or more, four times among five times, from the value of LF/HF calculated before the display.

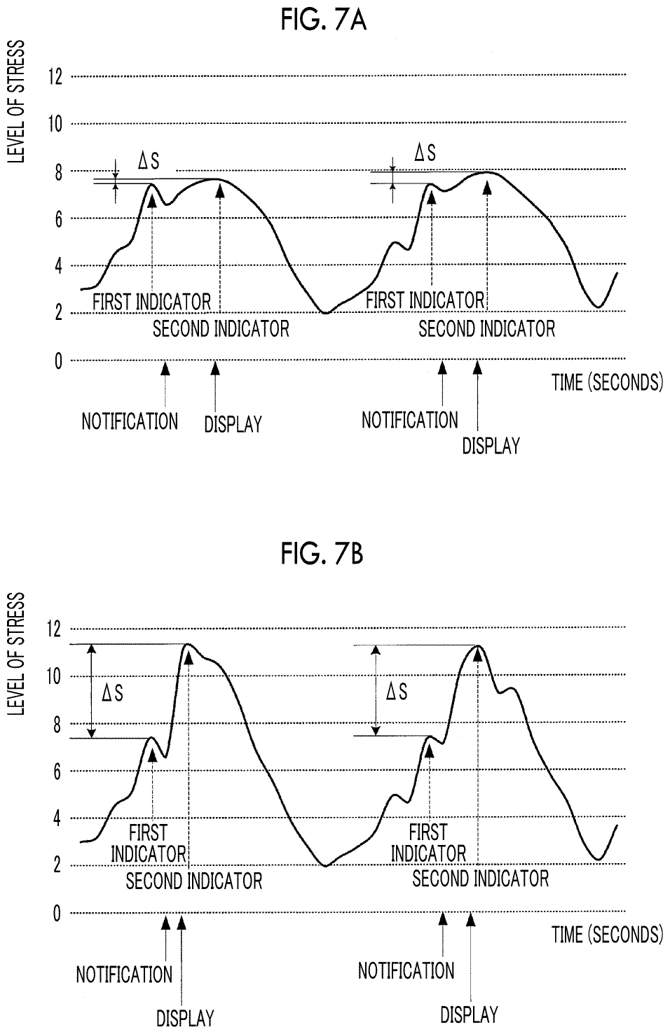

FIGS. 7A and 7B are diagrams illustrating fluctuations in the level of stress of the measurement target subject detected using the wearable terminal device 10. The vertical axes of FIGS. 7A and 7B each indicate the level of stress, and the horizontal axes each indicate the time. The peak values in FIGS. 7A and 7B each indicate that the measurement target subject is stressed.

In each of FIGS. 7A and 7B, the first peak value indicates the level of stress based on the value of LF/HF calculated from the biometric data detected before the level of stress as the first indicator is displayed. Then, the second peak value indicates the level of stress based on the value of LF/HF calculated from the biometric data detected immediately after the level of stress as the second indicator is displayed on the display screen of the portable terminal device 12 of the measurement target subject.

For example, a difference ΔS between an absolute value S2 of the value of LF/HF as the second indicator and an absolute value S1 of the value of LF/HF as the first indicator is calculated, and it is determined that the difference ΔS becomes equal to or greater than the predetermined value a predetermined number of times.

Then, in step S18, in a case where the value of LF/HF calculated after the display of the level of stress on the display screen does not change by the predetermined value or more the predetermined number of times from the value of LF/HF calculated before the display, the display mode of the portable terminal device 12 of the measurement target subject is not changed. That is, as illustrated in FIG. 7A, in a case where the difference ΔS becomes not greater than the predetermined value the predetermined number of times, the display mode is not changed. That is, by displaying the contents illustrated in FIG. 6 on the portable terminal device 12, it is not determined that the stress state of the measurement target subject deteriorates, and it is determined that there is no correlation between the display contents of FIG. 6 and the stress state of the measurement target subject. As a result, the display mode is not changed.

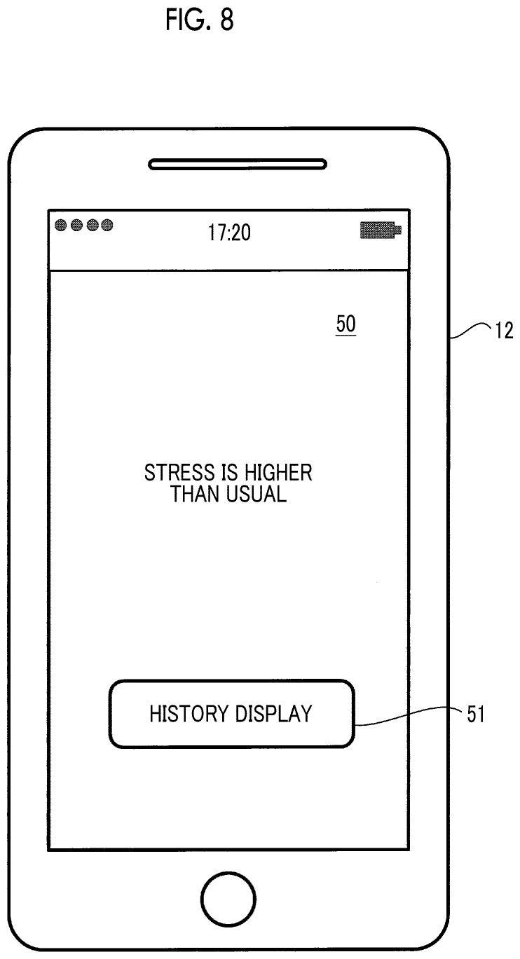

In step S19, in a case where the value of LF/HF calculated after the display of the level of stress on the display screen changes by the predetermined value or more the predetermined number of times from the value of LF/HF calculated before the display, the display control unit 44 changes the display mode of the portable terminal device 12 of the measurement target subject. That is, as illustrated in FIG. 7B, in a case where the difference ΔS becomes equal to or greater than the predetermined value the predetermined number of times, the display control unit 44 performs control for changing the display mode to be used next time onwards to, for example, such a display that does not deteriorate the stress state of the measurement target subject as illustrated in FIG. 8. In other words, by displaying the contents illustrated in FIG. 6 on the portable terminal device 12, it is determined that the stress state of the measurement target subject deteriorates, and it is determined that the display contents of FIG. 6 correlate with the stress state of the measurement target subject. As a result, the display mode is changed to, for example, display contents illustrated in FIG. 8.

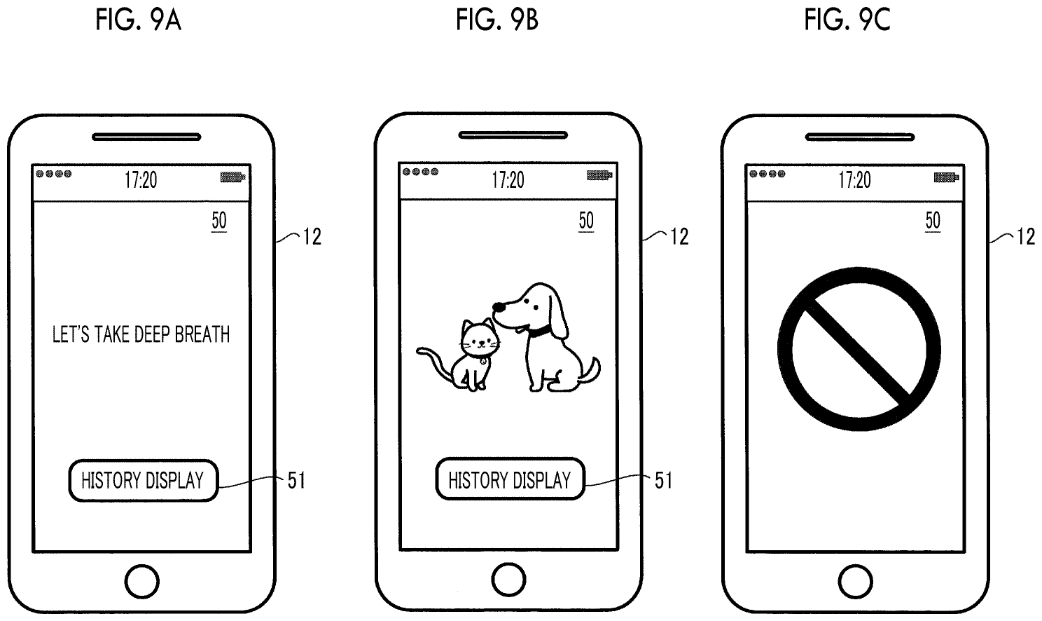

Next, another display screen example for changing the display mode will be described with reference to FIG. 9.

The value of LF/HF calculated after the display of the level of stress on the display screen may change by the predetermined value the predetermined number of times from the value of LF/HF calculated before the display. In this case, the display control unit 44 may perform control for changing the display mode of the portable terminal device 12 of the measurement target subject to: a display which prompts the measurement target subject to take an action for making the parasympathetic nervous system of the measurement target subject dominant as illustrated in FIG. 9A; a display such as a display of an animal image or a landscape image in which the parasympathetic nervous system of the measurement target subject is dominant as illustrated in FIG. 9B; a display in which the level of stress of the measurement target subject is not notified to the portable terminal device 12 or is less frequently notified with a half frequency as illustrated in FIG. 9C; or the like.

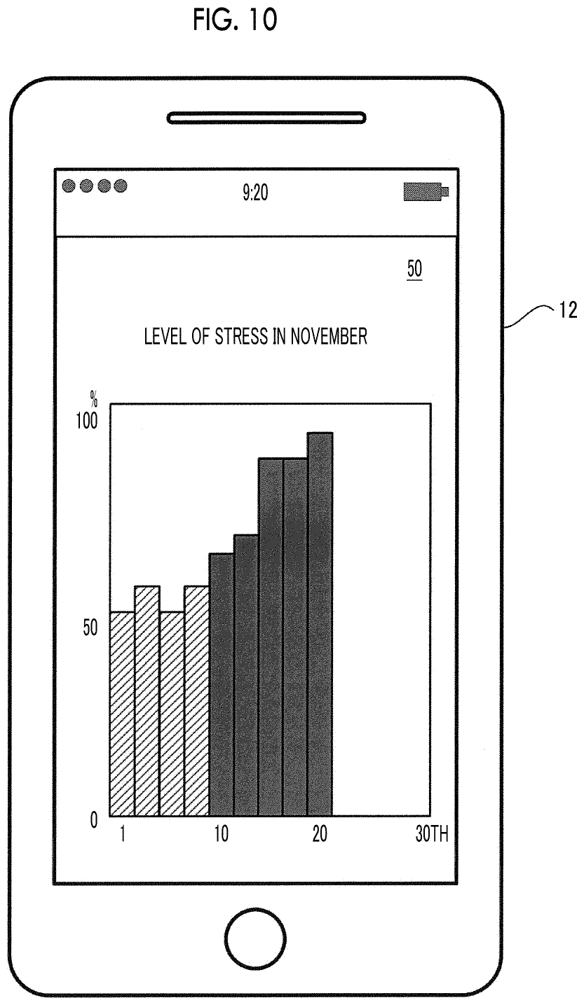

Next, a modification of the display screen indicating the level of stress of the measurement target subject in the portable terminal device 12 will be described with reference to FIG. 10.

In a case where a history display button 51 is pressed on the display screen of the portable terminal device 12 as illustrated in FIGS. 6 and 8 and the like, as illustrated in FIG. 10, the display control unit 44 performs control such that the current level of stress of the measurement target subject is displayed consecutively after the level of stress based on the plurality of previously calculated values of LF/HF. That is, the current level of stress of the measurement target subject is displayed together with the displacement of the previous level of stress. FIG. 10 shows an example in which the predetermined value of LF/HF is set to 100% stress, and the value of LF/HF of the measurement target subject is converted as a percentage so as to display the value as the level of stress of the measurement target subject. Further, in FIG. 10, the level of stress on each day is displayed in different color arrangement in accordance with the level of stress. For example, in a high stress state, the level of stress is displayed in color arrangement different from the low stress state and the medium stress state. Specifically, FIG. 10 shows that the measurement target subject continues to be in a high stress state since November 10. The level of stress may be displayed not on a date basis but on a time basis.

Then, immediately after the portable terminal device 12 is controlled to display the contents illustrated in FIG. 10 on the portable terminal device 12, the data acquisition unit 41 acquires the biometric data detected using the wearable terminal device 10, and the value of LF/HF is calculated from the acquired biometric data. Then, in a case where data of the value of LF/HF indicating the level of stress of the measurement target subject is accumulated, it is determined whether or not the value of LF/HF calculated after the display of the level of stress on the display screen changes by the predetermined value from the value of LF/HF calculated before the display of the value of LF/HF.

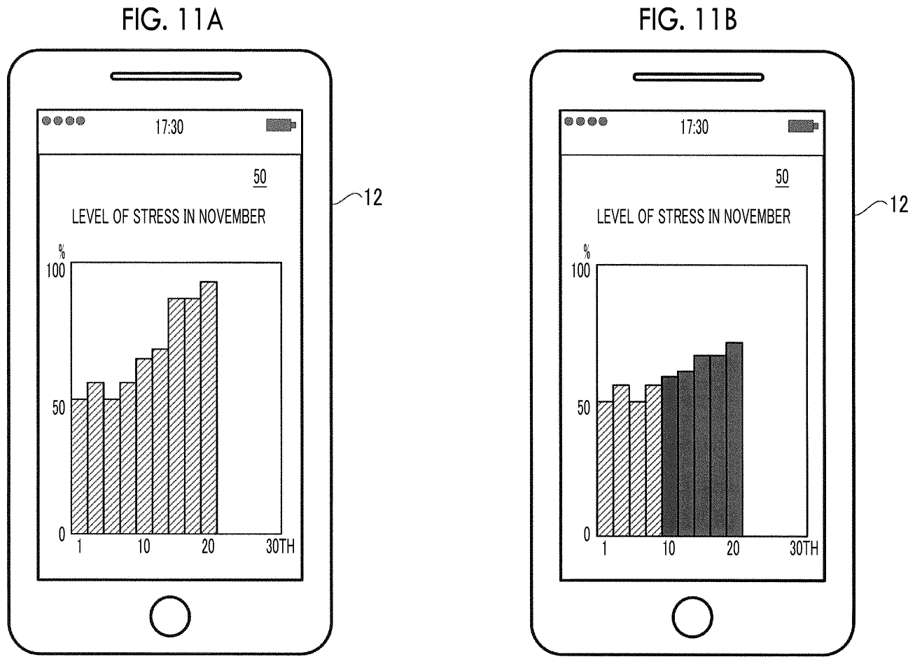

Then, in a case where the above-mentioned processing is repeated for a predetermined period and the value of LF/HF calculated after the display of the level of stress on the display screen changes by the predetermined value the predetermined number of times from the value of LF/HF calculated before the display, the display control unit 44 performs control for changing the display mode of the portable terminal device 12 of the measurement target subject. That is, the display control unit 44 performs control for changing the display mode to be used next time onwards to, for example, a display which makes deterioration in the stress state of the measurement target subject less noticeable such that the measurement target subject does not catch the detection result excessively and the stress state of the measurement target subject does not further deteriorates as illustrated in FIGS. 11A and 11B. In other words, by displaying the contents illustrated in FIG. 10 on the portable terminal device 12, it is determined that the stress state of the measurement target subject deteriorates, and it is determined that the display contents of FIG. 10 correlates with the stress state of the measurement target subject. As a result, the display mode is changed to, for example, display contents illustrated in FIGS. 11A and 11B.

Specifically, as illustrated in FIG. 11A, the display control unit 44 performs control for changing a display which makes deterioration in the stress state less noticeable by making coloration of the high stress state since November 10 in FIG. 10 the same as coloration of the stress state which is not a high stress state before November 10. Further, as illustrated in FIG. 11B, the display control unit 44 performs control for changing to a display which makes deterioration in the stress state less noticeable by reducing the scale of the graph in the high stress state since November 10 in FIG. 10.

Modification

The above embodiment has been described using an example in which the biometric data is detected using the wrist-type wearable terminal device 10. However, the present invention is not limited to this, and the present invention can be similarly applied to a case of using a wearable terminal device such as a clothing type, a band type wound around a wrist or an ankle, a glasses type, a hat type, or an adhesive patch type. Further, the present invention can be similarly applied to a case of detecting the biometric data using the image forming apparatus 13.

The above embodiment has been described using an example in which biometric data such as fluctuation in heart rate is detected as the biometric data of the measurement target subject and calculated based on the value of LF/HF. The present invention is limited to this, and the present invention can be similarly applied to a case of detecting the biometric data such as pulse, blood pressure, and brain waves. Further, the present invention can be similarly applied to even a case where biometric data of the measurement target subject is detected and a state of the measurement target subject such as a health state of the measurement target subject is used.

The above embodiment has been described using an example in which the level of stress calculated based on the value of LF/HF is displayed as an indicator for indicating the stress state of the measurement target subject. However, the value itself of LF/HF may be displayed as an indicator for indicating the stress state of the measurement target subject. That is, the indicator for indicating the stress state of the measurement target subject includes not only the level of stress calculated based on the value of LF/HF but also the value of LF/HF.

The above embodiment has been described using an example of control in which the biometric data of the measurement target subject detected by the wearable terminal device 10 is transmitted to the management server 14 through the Internet 18 by using the portable terminal device 12 and the detection result is displayed on the display screen of the portable terminal device 12 of the measurement target subject. However, this invention is not limited to this, and control may be performed such that the detection result is displayed on the display screen of the wearable terminal device 10 of the measurement target subject, the display screen of the image forming apparatus 13, or the display screen of the terminal device 15 of the measurement target subject or the manager. Further, the wearable terminal device 10 may be directly connected to the management server 14 by wireless communication such as Wi-Fi (registered trademark). In such a case, the present invention can be similarly applied to a case of performing control for displaying the detection result on the display screen of the wearable terminal device 10 of the measurement target subject. Further, the present invention can be similarly applied to a case of performing control in which the biometric data of the measurement target subject detected by the wearable terminal device 10 is transmitted to the management server 14 through the Internet 18 by using the image forming apparatus 13, and the detection result is displayed on the display screen of the wearable terminal device 10 of the measurement target subject, on the display screen of the image forming apparatus 13, or on the display screen of the terminal device 15 of the measurement target subject or the manager.

The embodiments of the present invention have been hitherto described in detail. However, the present invention is not limited to the above-mentioned embodiment, and various modifications can be made without departing from the scope of the invention.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A state display apparatus comprising:
a display section that displays a state of a measurement target subject based on biometric data of the measurement target subject; and
a control section that performs control such that a display mode at the time of displaying a state of the measurement target subject next time onwards is changed so as to be different from a display mode at the time of displaying a first state based on first biometric data of the measurement target subject, in a case where second biometric data acquired at the time of displaying the first state on the display section satisfies a predetermined condition, at the time of performing control such that the display section displays the first state.

2. The state display apparatus according to claim 1, further comprising:
a calculation section that calculates an indicator for indicating a corresponding state of the measurement target subject based on the biometric data acquired from the measurement target subject,
wherein the control section performs control such that the display mode at the time of displaying the state of the measurement target subject next time onwards is changed so as to be different from a display mode at the time of displaying a first indicator calculated based on the first biometric data in a case where a second indicator calculated based on the second biometric data changes by a predetermined value or more from the first indicator.

3. The state display apparatus according to claim 2,
wherein the control section performs control such that the display section does not display an indicator calculated next time onwards.

4. The state display apparatus according to claim 2,
wherein the control section performs control such that the display section displays the first indicator together with an indicator calculated based on biometric data acquired before the first biometric data is acquired.

5. The state display apparatus according to claim 2,
wherein the control section performs control such that the display section consecutively displays a plurality of indicators, which are calculated based on biometric data acquired before the first biometric data is acquired, and the first indicator.

6. The state display apparatus according to claim 4,
wherein the control section performs control such that the display section consecutively displays a plurality of indicators, which are calculated based on biometric data acquired before the first biometric data is acquired, and the first indicator.

7. The state display apparatus according to claim 4,
wherein the control section performs control for changing to a display in which deterioration in the state of the measurement target subject is less noticeable.

8. The state display apparatus according to claim 5,
wherein the control section performs control for changing to a display in which deterioration in the state of the measurement target subject is less noticeable.

9. The state display apparatus according to claim 6,
wherein the control section performs control for changing to a display in which deterioration in the state of the measurement target subject is less noticeable.

10. The state display apparatus according to claim 7,
wherein the display in which deterioration in the state of the measurement target subject is less noticeable includes a display in which the first indicator is displayed in the same color arrangement as the indicator calculated based on the biometric data acquired before the first biometric data is acquired.

11. The state display apparatus according to claim 8,
wherein the display in which deterioration in the state of the measurement target subject is less noticeable includes a display in which the first indicator is displayed in the same color arrangement as the indicator calculated based on the biometric data acquired before the first biometric data is acquired.

12. The state display apparatus according to claim 9,
wherein the display in which deterioration in the state of the measurement target subject is less noticeable includes a display in which the first indicator is displayed in the same color arrangement as the indicator calculated based on the biometric data acquired before the first biometric data is acquired.

13. The state display apparatus according to claim 10,
wherein the display in which deterioration in the state of the measurement target subject is less noticeable includes a display in which the first indicator is displayed with a scale at which a difference between the first indicator and the indicator calculated based on the biometric data acquired before the first biometric data is acquired is less noticeable.

14. The state display apparatus according to claim 11,
wherein the display in which deterioration in the state of the measurement target subject is less noticeable includes a display in which the first indicator is displayed with a scale at which a difference between the first indicator and the indicator calculated based on the biometric data acquired before the first biometric data is acquired is less noticeable.

15. The state display apparatus according to claim 12,
wherein the display in which deterioration in the state of the measurement target subject is less noticeable includes a display in which the first indicator is displayed with a scale at which a difference between the first indicator and the indicator calculated based on the biometric data acquired before the first biometric data is acquired is less noticeable.

16. The state display apparatus according to claim 1, further comprising:
a notification section that notifies a current state of the measurement target subject,
wherein at the time of performing the control such that the display section displays the first state based on first biometric data of the measurement target subject, in the case where the second biometric data acquired at the time of displaying the first state on the display section satisfies the predetermined condition, the control section performs control such that contents of notification at the time of notifying the state of the measurement target subject next time onwards are changed.

17. The state display apparatus according to claim 16,
wherein the control section performs control such that the contents of notification at the time of notifying the state of the measurement target subject next time onwards are changed to contents for prompting the measurement target subject to take action.

18. The state display apparatus according to claim 16,
wherein the control section performs control such that a frequency of the notification of the state of the measurement target subject, which is not issued by the notification section or is issued by the notification section next time onwards, is lowered.

19. A state display system comprising:
a detection section that detects biometric data of a measurement target subject;
an acquisition section that acquires the biometric data of the measurement target subject detected by the detection section;
a display section that displays a state of the measurement target subject acquired by the acquisition section; and
a control section that performs control such that a display mode at the time of displaying a state of the measurement target subject next time onwards is changed so as to be different from a display mode at the time of displaying a first state based on first biometric data of the measurement target subject, in a case where second biometric data acquired at the time of displaying the first state on the display section satisfies a predetermined condition, at the time of performing control such that the display section displays the first state.

20. A non-transitory computer readable medium storing a program causing a computer to execute:
acquiring biometric data of a measurement target subject;
displaying a state of the measurement target subject from the acquired biometric data; and
performing control such that a display mode at the time of displaying a state of the measurement target subject next time onwards is changed so as to be different from a display mode at the time of displaying a first state based on first biometric data of the measurement target subject, in a case where second biometric data acquired at the time of displaying the first state on the display section satisfies a predetermined condition, at the time of performing control such that the display section displays the first state.
